(19) United States
(12) Patent Application Publication
(10) Pub. No.: US 2023/0274844 A1
(43) Pub. Date: Aug. 31, 2023

(54) INFORMATION PROCESSING METHOD AND INFORMATION PROCESSING DEVICE

(71) Applicant: Fujitsu Limited, Kawasaki-shi (JP)

(72) Inventor: Yoshinobu IIMURA, Kawasaki (JP)

(73) Assignee: Fujitsu Limited, Kawasaki-shi (JP)

(21) Appl. No.: 18/065,001

(22) Filed: Dec. 13, 2022

(30) Foreign Application Priority Data
Feb. 25, 2022 (JP) 2022-028657

(51) Int. Cl. G16H 70/40

(52) U.S. Cl. CPC G16H 70/40

(57) ABSTRACT

A non-transitory computer-readable recording medium stores an information processing program for causing a computer to execute a process including generating a representative scenario representing a plurality of forecast scenarios, generating a representative model enabling to calculate a future value of a third variable by using a value of a first variable defined by the representative scenario and a value of a second variable, generating a deviation model representing a deviation between the representative model and a prediction model, identifying a mathematical expression enabling to calculate an analytical solution for a deviation of the second variable so as to optimize a value of a first objective variable, calculating a reference solution for the value of the second variable so as to optimize a value of a second objective variable, and calculating a numerical value solution for the value of the second variable, based on the reference solution and the analytical solution.

### CROSS-REFERENCE TO RELATED APPLICATION

This application is based upon and claims the benefit of priority of the prior Japanese Patent Application No. 2022-28657, filed on Feb. 25, 2022, the entire contents of which are incorporated herein by reference.

### FIELD

The embodiment discussed herein is related to an information processing method and an information processing device.

### BACKGROUND

Conventionally, there is a technique for optimizing a value of a control variable to be a future input so that a future output falls within a specific range, based on a forecast scenario that defines a value of a future explanatory variable in a specific period. For example, in the medical field, it is considered to optimize an insulin dosage to be a future input so that a blood sugar level of a subject to be a future output falls within a specific range, based on a forecast scenario that defines a change amount of a future blood sugar level of the subject, which is derived from meals, in a specific period.

As related art, for example, there is a technique for using a cooperation simulation of a physical model and a self-adoptive prediction controller using a hybrid automaton. Furthermore, for example, there is a technique for predicting future disturbance in model predictive control (MPC) applications by separating a transient part and a steady state value associated with the disturbance. Furthermore, for example, there is a technique for calculating a prediction value of an output of the control object by using a control object model for each of the plurality of control object models respectively created by using different values of the physical quantity. Furthermore, for example, there is a technique for predicting a control amount future value based on a dynamic characteristics model of the control object having one or more operation amounts and one or more control amounts.

U.S. Patent Application Publication No. 2020/0108203, U.S. Patent Application Publication No. 2011/0060424, Japanese Laid-open Patent Publication No. 2020-129292, and Japanese Laid-open Patent Publication No. 7-84610 are disclosed as related art. C. Noguchi et al., “In Silico Blood Glucose Control for Type 1 Diabetes with Meal Announcement Using Carbohydrate Intake and Glycemic Index”, Adv Biomed Eng. 5, pp. 124-131, 2016 is also disclosed as related art.

### SUMMARY

According to an aspect of the embodiment, a non-transitory computer-readable recording medium stores an information processing program for causing a computer to execute a process, the process includes generating a representative scenario that represents a plurality of forecast scenarios that defines a future value of a first variable, based on the plurality of forecast scenarios, generating a representative model that enables to calculate a future value of a third variable by using a value of the first variable that is defined by the generated representative scenario and a value of a second variable for control, generating, for each of the plurality of forecast scenarios, a deviation model that represents a deviation between the representative model and a prediction model that enables to calculate the future value of the third variable by using the value of the first variable that is defined by each of the plurality of forecast scenarios and the value of the second variable, identifying, for each generated deviation model, a mathematical expression that enables to calculate an analytical solution for a deviation of the second variable, by using each generated deviation model, so as to optimize a value of a first objective variable based on a deviation of the third variable, calculating a reference solution for the value of the second variable, by using the generated representative model and a constraint condition based on the deviation of the third variable that corresponds to an analytical solution that is able to be calculated from each identified mathematical expression, so as to optimize a value of a second objective variable based on the value of the third variable, and calculating a numerical value solution for the value of the second variable, based on the calculated reference solution and the analytical solution that is able to be calculated from each identified mathematical expression.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of an information processing method according to an embodiment;

FIG. 2 is a diagram illustrating an example of a blood sugar level management system;

FIG. 3 is a block diagram illustrating a hardware configuration example of an information processing device;

FIG. 4 is a block diagram illustrating a functional configuration example of the information processing device;

FIG. 5 is a diagram (part 1) illustrating an operation example of the information processing device;

FIG. 6 is a diagram (part 2) illustrating the operation example of the information processing device 100;

FIG. 7 is a diagram (part 3) illustrating the operation example of the information processing device;

FIG. 8 is a diagram illustrating another operation example of the information processing device; and

FIG. 9 is a flowchart illustrating an example of an overall processing procedure.

### DESCRIPTION OF EMBODIMENT

The relater art has a problem of increasing a processing load applied when a value of a control variable is optimized. For example, as the number of forecast scenarios is larger, the number of control variables of which the values are optimized increases, and this increases the processing load.

Hereinafter, an embodiment of an information processing method and an information processing device will be described in detail with reference to the drawings.

(Example of Information Processing Method According to Embodiment)

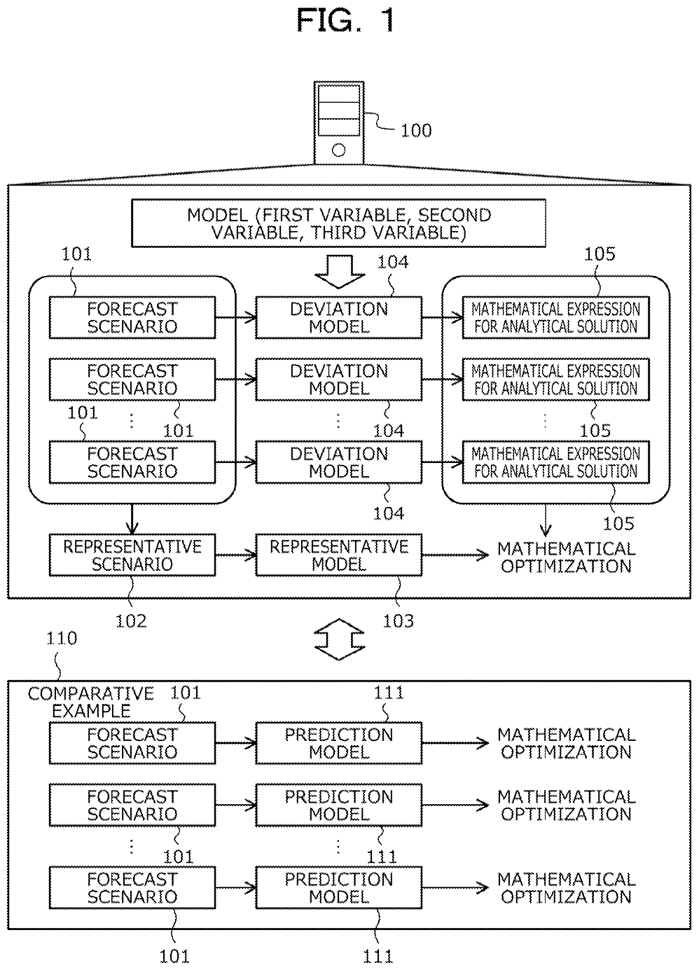

FIG. 1 is a diagram illustrating an example of an information processing method according to the embodiment. An information processing device 100 is a computer that reduces a processing load applied when a solution is calculated.

The solution is, for example, a value of a control variable to be a future input, that may cause a future output to fall within a specific range. The solution is a value of a control variable to be a future input, that may cause a future output to fall within the specific range when a forecast scenario is realized. The forecast scenario defines a value of a future explanatory variable. For example, it is desired to calculate a solution for each forecast scenario.

On the other hand, for each forecast scenario, a case is assumed where the forecast scenario is realized, and a method is considered for calculating a solution for a control variable to be a future input so as to cause a future output to fall within the specific range through mathematical optimization. However, this method has a problem of increasing the processing load applied when the solution for the control variable is calculated. For example, as the number of forecast scenarios is larger, the number of control variables for which calculation of the solutions are desired increases, and this increases the processing load applied when the solution is calculated.

For example, in the medical field, there is a case where it is desirable to perform automatic insulin dosing, with an assumption that a forecast scenario is realized, by calculating a solution for an insulin dosage to be a future input in advance so that a blood sugar level of a subject, to be a future output, falls within a specific range. The forecast scenario defines, for example, a change amount of a future blood sugar level of the subject, which is derived from meals. The specific range is, for example, a range of which a lower limit value is equal to or more than 80 so that life-threatening danger may be avoided. In this case, as the number of forecast scenarios is larger, the processing load applied when the solution is calculated increases. Therefore, there is a problem in that it is difficult to perform automatic insulin dosing in real time.

In the present embodiment, an information processing method will be described that may reduce a processing load applied when a solution is calculated.

The information processing device 100 stores a plurality of forecast scenarios 101 that defines a future value of a first variable. The value of the first variable is, for example, a change amount of a blood sugar level of a subject according to meals. The plurality of forecast scenarios 101 respectively define change amounts of a blood sugar level of the subject according to meals in different patterns. The pattern is, for example, a combination of a meal amount and a meal time point.

(1-1) The information processing device 100 generates a representative scenario 102 based on the plurality of forecast scenarios 101. The representative scenario 102 represents the plurality of forecast scenarios 101. The information processing device 100 generates, for example, the representative scenario 102 that defines an average value of the first variables in the plurality of forecast scenarios 101.

(1-2) The information processing device 100 generates a representative model 103. The representative model 103 enables to calculate a future value of a third variable using the value of the first variable defined by the generated representative scenario 102 and a value of a second variable for control. The representative model 103 is, for example, a function that enables to calculate a value of the third variable at a next time point, based on a value of the first variable, a value of the second variable, and a value of the third variable at a certain time point. The value of the second variable is, for example, an insulin dosage to the subject. The value of the third variable is, for example, related to the blood sugar level of the subject. The value of the third variable is, for example, a difference indicating the blood sugar level of the subject, with 100 as a reference. That is, the value of the third variable is (blood sugar level of subject—100).

(1-3) The information processing device 100 generates a deviation model 104, for each of the plurality of forecast scenarios 101. The deviation model 104 represents a deviation between a prediction model 111, which enables to calculate the future value of the third variable using the value of the first variable and the value of the second variable both defined by the forecast scenario 101, and the representative model 103. The deviation model 104 is, for example, a function that enables to calculate a deviation of the third variable at a next time point, based on a deviation of the first variable, a deviation of the second variable, and a deviation of the third variable at a certain time point, with the representative model 103 as a reference.

(1-4) The information processing device 100 identifies, for each generated deviation model 104, a mathematical expression 105 that enables to calculate an analytical solution for the deviation of the second variable, using the deviation model 104, so as to optimize a value of a first objective variable based on the deviation of the third variable. The value of the first objective variable is, for example, defined based on a deviation with the representative model 103 as a reference, of a difference between a reference value and the blood sugar level of the subject. The first objective variable includes, for example, a square of the deviation with the representative model 103 as a reference, of the difference between the reference value and the blood sugar level of the subject. The value of the first objective variable is, for example, a target to be minimized. A processing load that is applied when the mathematical expression 105 is identified and the analytical solution is calculated tends to be relatively small.

(1-5) The information processing device 100 calculates a reference solution for the value of the second variable, using the generated representative model 103 and a constraint condition so as to optimize a value of a second objective variable based on the value of the third variable. The value of the second objective variable is, for example, defined based on the difference between the reference value and the blood sugar level of the subject. The second objective variable includes, for example, a square of the difference between the reference value and the blood sugar level of the subject. The value of the second objective variable is, for example, a target to be minimized.

The constraint condition is based on the deviation of the third variable corresponding to the analytical solution that may be calculated from each identified mathematical expression 105. The constraint condition indicates, for example, that a blood sugar level of the subject obtained by adding a reference of the blood sugar level of the subject corresponding to the reference solution and a minimum value of the deviations of the blood sugar level of the subject corresponding to the analytical solutions that may be calculated from the respective mathematical expressions 105 is equal to or more than a lower limit value. The information processing device 100 calculates the reference solution for the value of the second variable through mathematical optimization. It is sufficient for the information processing device 100 to perform mathematical optimization once regardless of the number of forecast scenarios 101.

(1-6) The information processing device 100 calculates a numerical value solution for the value of the second variable, based on the calculated reference solution and the analytical solution that may be calculated from each identified mathematical expression 105. The information processing device 100 calculates the numerical value solution for each forecast scenario 101, for example, by adding the calculated reference solution and the analytical solution that may be calculated from each identified mathematical expression 105. As a result, since the information processing device 100 only needs to perform mathematical optimization once regardless of the number of forecast scenarios 101, it is possible to reduce a processing load that is applied when the numerical value solution for each forecast scenario 101 is calculated.

Conventionally, as indicated in a comparative example 110, a method is considered for using a prediction model 111 corresponding to each forecast scenario 101, and calculating a numerical value solution for each forecast scenario 101 through mathematical optimization. With this method, as the number of forecast scenarios 101 is larger, the number of times of mathematical optimization increases, and this increases a processing load applied when the numerical value solution for each forecast scenario is calculated. Whereas the information processing device 100 may reduce the processing load applied when the numerical value solution for each forecast scenario is calculated.

Here, a case where the information processing device 100 operates alone has been described. However, the embodiment is not limited to this. For example, a plurality of computers may cooperate to implement functions as the information processing device 100 and perform the operations as the information processing device 100.

(Example of Blood Sugar Level Management System 200)

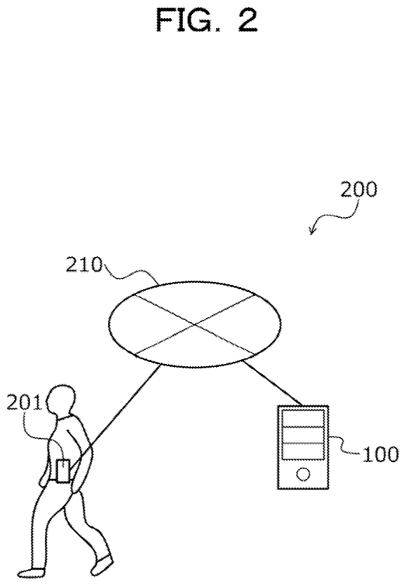

Next, an example of a blood sugar level management system 200 to which the information processing device 100 illustrated in FIG. 1 is applied will be described with reference to FIG. 2.

FIG. 2 is a diagram illustrating an example of the blood sugar level management system 200. In FIG. 2, the blood sugar level management system 200 includes the information processing device 100 and a terminal device 201.

In the blood sugar level management system 200, the information processing device 100 and the terminal device 201 are coupled via a wired or wireless network 210. The network 210 is, for example, a local area network (LAN), a wide area network (WAN), the Internet, or the like.

The information processing device 100 is a computer that controls an insulin dosage to the subject. The information processing device 100 stores a plurality of forecast scenarios that define change amounts of the blood sugar level of the subject according to meals respectively in different patterns. The information processing device 100 generates a representative scenario based on the plurality of forecast scenarios.

The information processing device 100 generates a representative model for the generated representative scenario. The representative model corresponds to, for example, a case where the representative scenario is realized. For example, the representative model enables to calculate (blood sugar level of subject—100) at a next time point, based on a change amount of the blood sugar level of the subject according to meals, an insulin dosage, and (blood sugar level of subject—100) at a certain time point.

The information processing device 100 generates a deviation model for each of the plurality of forecast scenarios. The deviation model corresponds to a deviation between the representative model and a prediction model corresponding to the forecast scenario. The prediction model corresponds to, for example, a case where the forecast scenario is realized. For example, the prediction model enables to calculate (blood sugar level of subject—100) at a next time point, based on a change amount of the blood sugar level of the subject according to meals, an insulin dosage, and (blood sugar level of subject— 100) at a certain time point.

For example, the deviation model enables to calculate a deviation of (blood sugar level of subject—100) at a next time point, based on a deviation of the change amount of the blood sugar level of the subject according to meals, a deviation of the insulin dosage, and a deviation of (blood sugar level of subject—100) at a certain time point. The deviation of the change amount is, for example, a deviation between a change amount in a case where the representative scenario is realized and a change amount in a case where the forecast scenario is realized. The deviation of the insulin dosage is, for example, a deviation between an insulin dosage in a case where the representative scenario is realized and an insulin dosage in a case where the forecast scenario is realized. The deviation of (blood sugar level of subject—100) is, for example, a deviation between (blood sugar level of subject—100) in a case where the representative scenario is realized and (blood sugar level of subject—100) in a case where the forecast scenario is realized.

The information processing device 100 identifies a mathematical expression that enables to calculate an analytical solution for the deviation of the insulin dosage, using the deviation model, so as to optimize a value of a first objective variable based on the deviation of (blood sugar level of subject—100) for each generated deviation model. The first objective variable includes, for example, a square of the deviation of (blood sugar level of subject—100). The first objective variable may include, for example, a square of the deviation of the insulin dosage. The first objective variable is, for example, a target to be minimized.

The information processing device 100 calculates a reference solution for the insulin dosage, using the generated representative model and a constraint condition, so as to optimize a value of a second objective variable based on (blood sugar level of subject—100), through mathematical optimization. The second objective variable includes, for example, a square of (blood sugar level of subject—100). The second objective variable may include, for example, a square of the insulin dosage. The second objective variable is, for example, a target to be minimized.

The constraint condition indicates, for example, that a blood sugar level of the subject obtained by adding a reference of the blood sugar level corresponding to the reference solution and a minimum value of the deviations of the blood sugar level of the subject corresponding to the analytical solutions that may be calculated from the respective mathematical expressions is equal to or more than a lower limit value. The information processing device 100 calculates a numerical value solution for a future insulin dosage, for each forecast scenario, based on the calculated reference solution and the analytical solution that may be calculated from each identified mathematical expression.

The information processing device 100 transmits the numerical value solution for the future insulin dosage calculated for each forecast scenario, to the terminal device 201, in association with the forecast scenario. For example, the information processing device 100 is a server, a personal computer (PC), or the like.

The terminal device 201 is a computer that implements an insulin pump. The terminal device 201 receives the numerical value solution for the future insulin dosage from the information processing device 100, for each forecast scenario. The terminal device 201 controls an insulin dosage to the subject according to the realized forecast scenario, based on the numerical value solution for the insulin dosage. The terminal device 201 is, for example, a microcontroller or the like.

(Another Application Example of Information Processing Device 100)

The information processing device 100 may be applied to the electric field, for example. For example, it is considered that the information processing device 100 controls a discharge amount of a secondary battery not to be equal to or more than a demand power, according to a forecast scenario regarding the demand power. The information processing device 100 may be applied to, for example, the power generation field. For example, it is considered that the information processing device 100 controls a rotation speed of a windmill not to be equal to or more than a threshold that damages the windmill, according to a forecast scenario regarding a wind speed or the like.

(Hardware Configuration Example of Information Processing Device 100)

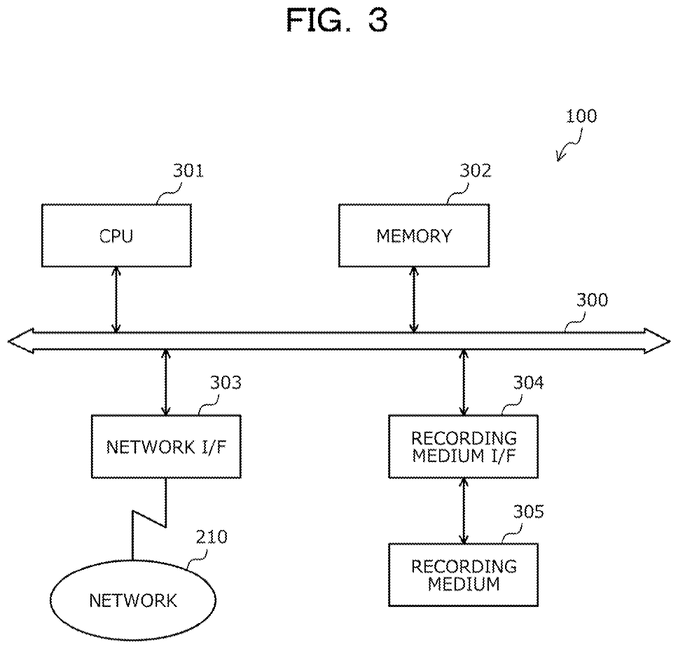

Next, a hardware configuration example of the information processing device 100 will be described with reference to FIG. 3.

FIG. 3 is a block diagram illustrating the hardware configuration example of the information processing device 100. In FIG. 3, the information processing device 100 includes a central processing unit (CPU) 301, a memory 302, a network interface (I/F) 303, a recording medium I/F 304, and a recording medium 305. Furthermore, the components are coupled to each other by a bus 300.

Here, the CPU 301 performs overall control of the information processing device 100. The memory 302 includes, for example, a read only memory (ROM), a random access memory (RAM), a flash ROM, or the like. For example, the flash ROM or the ROM stores various programs, and the RAM is used as a work area for the CPU 301. The program stored in the memory 302 is loaded into the CPU 301 to cause the CPU 301 to execute coded processing.

The network I/F 303 is coupled to the network 210 through a communication line and is coupled to another computer via the network 210. Then, the network I/F 303 manages an interface between the network 210 and the inside of the information processing device 100, and controls input and output of data from and to another computer. For example, the network I/F 303 is a modem, a LAN adapter, or the like.

The recording medium I/F 304 controls read and write of data from and to the recording medium 305 under the control of the CPU 301. The recording medium I/F 304 is, for example, a disk drive, a solid state drive (SSD), a universal serial bus (USB) port, or the like. The recording medium 305 is a nonvolatile memory that stores data written under the control of the recording medium I/F 304. For example, the recording medium 305 is a disk, a semiconductor memory, a USB memory, or the like. The recording medium 305 may be attachable to and detachable from the information processing device 100.

For example, the information processing device 100 may include a keyboard, a mouse, a display, a printer, a scanner, a microphone, a speaker, or the like in addition to the components described above. Furthermore, the information processing device 100 may include a plurality of the recording medium I/Fs 304 and recording media 305. Furthermore, the information processing device 100 does not have to include the recording medium I/F 304 and the recording medium 305.

(Hardware Configuration Example of Terminal Device 201)

A hardware configuration example of the terminal device 201 is, for example, similar to the hardware configuration example of the information processing device 100 illustrated in FIG. 3. The terminal device 201 further includes a mechanism to dose the subject with insulin. The terminal device 201 may further include a sensor that measures biological information of the subject. The biological information is, for example, a body temperature, a heart rate, a pulse, a blood pressure, a blood sugar level, or the like.

(Functional Configuration Example of Information Processing Device 100)

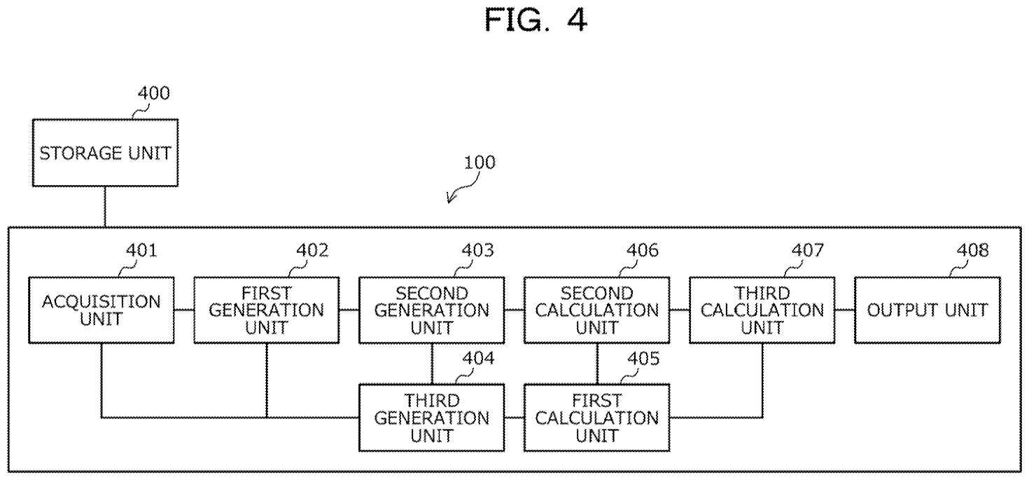

Next, a functional configuration example of the information processing device 100 will be described with reference to FIG. 4.

FIG. 4 is a block diagram illustrating the functional configuration example of the information processing device 100. The information processing device 100 includes a storage unit 400, an acquisition unit 401, a first generation unit 402, a second generation unit 403, a third generation unit 404, a first calculation unit 405, a second calculation unit 406, a third calculation unit 407, and an output unit 408.

The storage unit 400 is implemented by a storage area such as the memory 302 or the recording medium 305 illustrated in FIG. 3, for example. Hereinafter, a case where the storage unit 400 is included in the information processing device 100 will be described. However, the embodiment is not limited to this. For example, there may be a case where the storage unit 400 is included in a device different from the information processing device 100, and storage content of the storage unit 400 may be referred from the information processing device 100.

The acquisition unit 401 through the output unit 408 function as an example of a control unit. For example, the acquisition unit 401 through the output unit 408 implement functions thereof by causing the CPU 301 to execute a program stored in the storage area such as the memory 302 or the recording medium 305 illustrated in FIG. 3 or by the network I/F 303. A processing result of each functional unit is, for example, stored in the storage area such as the memory 302 or the recording medium 305 illustrated in FIG. 3.

The storage unit 400 stores various types of information to be referred or updated in processing of each functional unit. The storage unit 400 stores a plurality of forecast scenarios. Each of the forecast scenarios defines, for example, a future value of a first variable. The value of the first variable is, for example, a change amount of a blood sugar level of a subject according to meals. The value of the first variable may be, for example, a power demand amount or the like. The forecast scenario is acquired by the acquisition unit 401, for example. The forecast scenarios may be, for example, stored in the storage unit 400 in advance.

The storage unit 400 stores a representative scenario representing the plurality of forecast scenarios. The representative scenario defines, for example, an average value of the first variables in the plurality of forecast scenarios. The representative scenario defines, for example, a minimum value of the first variables in the plurality of forecast scenarios. The representative scenario is generated, for example, by the first generation unit 402.

The storage unit 400 stores a representative model. For example, the representative model enables to calculate a future value of a third variable using the value of the first variable defined by the representative scenario and a value of a second variable for control. The value of the second variable is, for example, an insulin dosage to the subject. The value of the second variable may be, for example, a power storage amount. The value of the third variable is, for example, a difference between a reference value and the blood sugar level of the subject. The reference value is, for example, 100. The value of the third variable may be a power charge amount. In a case where the power charge amount is a negative value, the power charge amount indicates a discharge amount. The representative model is generated, for example, by the second generation unit 403.

The storage unit 400 stores a deviation model. The deviation model represents a deviation between a prediction model that enables to calculate the future value of the third variable using the value of the first variable defined by the forecast scenario and the value of the second variable and the representative model. The deviation model enables to calculate a future deviation of the third variable, for example, using the deviation of the first variable and the deviation of the second variable for control. The deviation model is generated, for example, by the third generation unit 404.

The acquisition unit 401 acquires various types of information to be used for processing of each functional unit. The acquisition unit 401 stores the acquired various types of information in the storage unit 400, or outputs the acquired information to each functional unit. Furthermore, the acquisition unit 401 may output the various types of information stored in the storage unit 400 to each functional unit. The acquisition unit 401 acquires the various types of information, for example, on the basis of an operation input of a user. The acquisition unit 401 may receive the various types of information, for example, from a device different from the information processing device 100.

The acquisition unit 401 acquires the plurality of forecast scenarios. The acquisition unit 401 acquires, for example, the plurality of forecast scenarios by receiving the forecast scenarios from another computer. The another computer is, for example, the terminal device 201. The acquisition unit 401 may acquire the plurality of forecast scenarios, for example, by accepting inputs of the plurality of forecast scenarios based on the operation input of the user.

The acquisition unit 401 may accept a start trigger for starting processing of any one of the functional units. The start trigger is, for example, a predetermined operation input by the user. The start trigger may be, for example, reception of predetermined information from another computer. The start trigger may be, for example, an output of predetermined information by any functional unit. For example, the acquisition unit 401 may accept the acquisition of the plurality of forecast scenarios as the start trigger used to start processing of the first generation unit 402, the second generation unit 403, the third generation unit 404, the first calculation unit 405, the second calculation unit 406, and the third calculation unit 407.

The first generation unit 402 generates the representative scenario based on the plurality of forecast scenarios. The first generation unit 402 generates, for example, the representative scenario that defines a statistical value of the first variables in the plurality of forecast scenarios, based on the value of the first variable defined by each forecast scenario. The statistical value is, for example, a maximum value, a minimum value, an average value, a median, a mode, or the like.

The first generation unit 402 generates, for example, the representative scenario that defines the average value of the first variables in the plurality of forecast scenarios. As a result, for example, if the value of the first variable is the change amount of the blood sugar level of the subject, the first generation unit 402 enables the second calculation unit 406 to calculate the reference solution for the insulin dosage so that the blood sugar level of the subject does not rise too much.

The first generation unit 402 generates, for example, the representative scenario that defines the minimum value of the first variables in the plurality of forecast scenarios. As a result, the first generation unit 402 may easily satisfy the constraint condition. For example, if the value of the first variable is the change amount of the blood sugar level of the subject, the first generation unit 402 enables the second calculation unit 406 to calculate the reference solution for the insulin dosage so that it is difficult for the blood sugar level of the subject to fall below the lower limit value.

The second generation unit 403 generates the representative model for the generated representative scenario. The second generation unit 403 generates, for example, the representative model that enables to calculate the future value of the third variable, using the value of the first variable corresponding to the representative scenario and the value of the second variable for control. As a result, the second generation unit 403 enables to calculate the reference solution to be a reference for calculating the numerical value solution for the second variable.

The third generation unit 404 generates the deviation model for each of the plurality of forecast scenarios. For example, the third generation unit 404 generates the deviation model that enables to calculate the future deviation of the third variable, using the deviation of the first variable corresponding to the forecast scenario and the deviation of the second variable for control, for each forecast scenario. As a result, the third generation unit 404 may consider each forecast scenario, when the numerical value solution for the second variable is calculated, without depending on mathematical optimization.

The first calculation unit 405 identifies a mathematical expression that enables to calculate an analytical solution for the deviation of the second variable, using the deviation model, so as to optimize a value of a first objective variable for each generated deviation model. The value of the first objective variable is based on the deviation of the third variable. For example, if the deviation of the third variable is the deviation of (blood sugar level of subject—100), the value of the first objective variable includes a square of the deviation of (blood sugar level of subject—100) and is a target to be minimized. The value of the first objective variable may be, for example, further based on the deviation of the second variable. For example, if the deviation of the second variable is the deviation of the insulin dosage, the value of the first objective variable includes a square of the deviation of the insulin dosage.

As a result, the first calculation unit 405 may enable to calculate the analytical solution for the deviation of the second variable. For example, the first calculation unit 405 may enable to calculate an analytical solution for the deviation of the insulin dosage so that the deviation of (blood sugar level of subject—100) approaches zero. For example, the first calculation unit 405 may enable to calculate the analytical solution for the deviation of the insulin dosage, so that both of the deviation of (blood sugar level of subject—100) and the deviation of the insulin dosage approach zero.

The second calculation unit 406 calculates a reference solution for the value of the second variable using the generated representative model and a constraint condition based on the deviation of the third variable corresponding to the analytical solution that may be calculated from each identified mathematical expression so as to optimize a value of a second objective variable based on the value of the third variable. The constraint condition is, for example, a condition that is desired to be satisfied by a combination of the reference solution and the analytical solution that may be calculated from any one of mathematical expressions.

The constraint condition indicates, for example, that a blood sugar level of the subject obtained by adding a reference of the blood sugar level of the subject corresponding to the reference solution and a minimum value of the deviations of the blood sugar level of the subject corresponding to the analytical solutions that may be calculated from the respective mathematical expressions is equal to or more than a lower limit value. The constraint condition indicates, for example, that a value obtained by adding a reference of a power charge amount corresponding to the reference solution and a minimum value of deviations of the power charge amount corresponding to the analytical solutions that may be calculated from the respective mathematical expressions is equal to or more than the lower limit value.

For example, if the value of the third variable is (blood sugar level of subject—100), the value of the second objective variable includes a square of (blood sugar level of subject—100) and is a target to be minimized. The value of the first objective variable may be, for example, further based on the value of the second variable. For example, if the value of the second variable is the insulin dosage, the value of the first objective variable includes a square of the insulin dosage. As a result, the second calculation unit 406 may calculate the reference solution to be a reference used to calculate the numerical value solution for the second variable, so as to satisfy the constraint condition. The second calculation unit 406 may calculate the reference solution to a reference used to calculate a numerical value solution for the insulin dosage, for example, so that the blood sugar level of the subject is maintained to be equal to or more than the lower limit value, for each forecast scenario.

The third calculation unit 407 calculates the numerical value solution for the value of the second variable based on the calculated reference solution and the analytical solution that may be calculated from each identified mathematical expression. The third calculation unit 407 calculates the numerical value solution for the value of the second variable by adding the calculated reference solution and the analytical solution that may be calculated from the identified mathematical expression for each forecast scenario. As a result, the third calculation unit 407 may reduce the processing load applied when the numerical value solution for the value of the second variable is calculated.

The third calculation unit 407 may calculate a value of a fourth variable based on the numerical value solution calculated for the value of the second variable. The fourth variable is, for example, the blood sugar level of the subject. The third calculation unit 407 may calculate the blood sugar level of the subject, for example, based on the numerical value solution of the insulin dosage. As a result, the third calculation unit 407 may allow the user to refer to the value of the fourth variable.

The output unit 408 outputs a processing result of at least any one of the functional units. An output format is, for example, display on a display, print output to a printer, transmission to an external device by the network I/F 303, or storage in a storage area such as the memory 302 or the recording medium 305. Therefore, the output unit 408 may notify the user of the processing result of at least any functional unit and may improve convenience of the information processing device 100.

The output unit 408 outputs, for example, the numerical value solution calculated for the value of the second variable in association with each forecast scenario. For example, the output unit 408 transmits the numerical value solution calculated for the value of the second variable to another computer in association with each forecast scenario. The another computer is, for example, the terminal device 201. As a result, the output unit 408 may allow another computer to appropriately control the value of the second variable according to the realized forecast scenario.

The output unit 408 may output, for example, the calculated value of the fourth variable in association with each forecast scenario. For example, the output unit 408 transmits the calculated value of the fourth variable in association with each forecast scenario to another computer. The another computer is, for example, the terminal device 201. As a result, the output unit 408 may allow the user to refer to the value of the fourth variable corresponding to the realized forecast scenario.

(Operation Example of Information Processing Device 100)

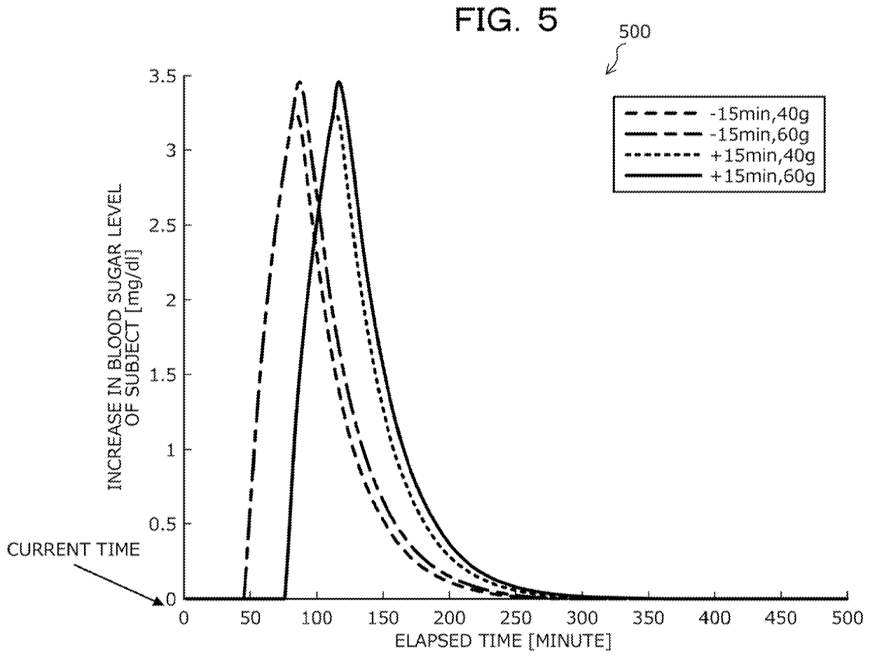

Next, an operation example of the information processing device 100 will be described with reference to FIGS. 5 to 7.

FIGS. 5 to 7 are diagrams illustrating the operation example of the information processing device 100. In FIG. 5, the information processing device 100 acquires a plurality of meal scenarios to be forecast scenarios f. In the example in FIG. 5, the information processing device 100 acquires, for example, four meal scenarios. A meal scenario indicates an increase [mg/dl] of a blood sugar level of a subject who has eaten a meal with respect to an elapsed time [minute]. The blood sugar level is also referred to as a blood glucose level.

One meal scenario corresponds to, for example, a case where a meal of a reference meal amount +10 g is consumed at a time that is +15 minutes of a reference time. The reference meal amount is, for example, 50 g. One meal scenario corresponds to, for example, a case where a meal of the reference meal amount +10 g is consumed at a time that is −15 minutes of the reference time. One meal scenario corresponds to, for example, a case where a meal of the reference meal amount −10 g is consumed at a time that is +15 minutes of the reference time. One meal scenario corresponds to, for example, a case where a meal of the reference meal amount −10 g is consumed at a time that is −15 minutes of the reference time.

A graph 500 illustrates an increase [mg/dl] in a blood sugar level of a subject who has consumed a meal with respect to an elapsed time [minute] in a case where each meal scenario is realized. The vertical axis of the graph 500 indicates the increase [mg/dl] in the blood sugar level of the subject. The horizontal axis of the graph 500 indicates the elapsed time [minute]. In the following description, in a case where the forecast scenarios f are distinguished from each other, an i-th forecast scenario f may be expressed as a “forecast scenario fⁱ”. Here, it is desirable to calculate an insulin dosage so that the blood sugar level of the subject falls within an allowable range regardless of which meal scenario is realized.

A relationship between an insulin dosage uₖ, an element xₖ including the blood sugar level of the subject, and a blood sugar level increase value fₖ indicated by a forecast scenario f at a time point k is defined, for example, by the following formula (1). The element xₖ includes, for example, subcutaneous depot insulin levels, subcutaneous absorption sections, plasma insulin concentrations, remote insulin concentrations, blood sugar levels, or the like. The element xₖ may include (blood sugar level of subject—100). The element xₖ₊₁ corresponds to a next time point k+1. D represents, for example, a basal blood sugar level. A, B, and B_{w} are coefficient matrices.

xₖ₊₁=Axₖ+Buₖ+B_{w}fₖ+D (1)

The information processing device 100 generates a representative scenario f- representing the plurality of forecast scenarios f, based on the plurality of forecast scenarios f. Here, for convenience, an “arbitrary character” to which “-” is added at the top is expressed in a form of “arbitrary character-”. For example, the information processing device 100 generates the representative scenario f- that defines an average value [mg/dl] of the increases of the blood sugar level indicated by the plurality of forecast scenarios f with respect to the elapsed time [minutes], according to the following formula (2).

f̅=(f¹++f²+f³+f⁴)/4 (2)

The information processing device 100 refers to the above formula (1) and generates a representative model defined by the following formula (3) using the representative scenario f-. The following formula (3) indicates a relationship between an insulin dosage u-ₖ, an element x-ₖ including the blood sugar level of the subject, and an increase f-ₖ of a blood sugar level indicated by the forecast scenario f-.

x̅ₖ₊₁=Ax̅ₖ+Būₖ+B_{w}f̅ₖ+D (3)

The information processing device 100 generates a deviation model defined by each of the following formulas (5) to (8) by referring to the above formula (1) and dividing, using each forecast scenario fⁱ, the prediction model defined by the following formula (4) into the generated representative model and the deviation model. The following formula (4) indicates a relationship between an insulin dosage uₖⁱ, an element xₖⁱ including the blood sugar level of the subject, and an increase fₖⁱ of a blood sugar level indicated by the forecast scenario fⁱ.

The deviation model corresponds to a deviation between the representative model and the prediction model. The following formulas (5) to (8) indicate a relationship between a deviation u˜ₖⁱ of the insulin dosage, a deviation x˜ₖⁱ of the element including the blood sugar level of the subject, and a deviation f˜ₖⁱ=(fₖⁱ−f₋ₖ) of the increase in the blood sugar level indicated by the forecast scenario fⁱ. A relationship between x− and x˜ is defined by the following formula (9).

xₖ₊₁=Axₖⁱ+Buₖⁱ+B_{w}fₖⁱ+D (4)

\tilde{x}ₖ₊₁¹=A\tilde{x}ₖ¹+Bũₖ¹+B_{w}(fₖ¹−f̅ₖ) (5)

\tilde{x}ₖ₊₁²=A\tilde{x}ₖ²+Bũₖ²+Bũₖ²+B_{w}(fₖ²−f̅ₖ) (6)

\tilde{x}ₖ₊₁³=A\tilde{x}ₖ³+Bũₖ³+Bũₖ³+B_{w}(fₖ³−f̅ₖ) (7)

\tilde{x}ₖ₊₁⁴=A\tilde{x}ₖ⁴+Bũₖ⁴+Bũₖ⁴+B_{w}(fₖ⁴−f̅ₖ) (8)

x=x̅+\tilde{x} (9)

The information processing device 100 calculates an analytical solution u˜ₖ* of the insulin dosage using a constraint condition defined by the following formula (11), so as to minimize an objective function indicated by the following formula (10), for each deviation model. The analytical solution u˜k* is defined by the following formula (12). Q˜, R˜, and S˜ are coefficient matrices. Here, a deviation y˜ₖi* of the blood sugar level at the time point k for the i-th forecast scenario fⁱ is defined by the following formula (13) using the analytical solution u˜ₖi*. The information processing device 100 may calculate the analytical solution u˜kⁱ* without depending on mathematical optimization. Therefore, the information processing device 100 may calculate the analytical solution u˜kⁱ* with a relatively small processing load.

$$min {{\sum }^{N}}_{k = 1} {\tilde{x}}_{k}^{T} \tilde{Q} {\tilde{x}}_{k} + {\tilde{u}}_{k}^{T} \tilde{R} {\tilde{u}}_{k} + {\tilde{x}}_{N + 1}^{T} {\tilde{S}}_{f} {\tilde{x}}_{N + 1} \qquad ( 10 )$$

\tilde{x}ₖ₊₁=A\tilde{x}ₖ+Bũₖ*+B_{w}fₖⁱ (11)

ũₖ*=−(Rₖ+B^{T}Sₖ₊₁B)⁻¹B^{T}(Sₖ₊₁(A\tilde{x}ₖ+B_{w}fₖⁱ)+Tₖ₊₁) (12)

\tilde{x}ₖ₊₁^{i*}=A\tilde{x}ₖ^{i*}+Bũₖ^{i*}+(fₖⁱ−f̅ₖ),\tilde{y}ₖ^{i*} (13)

The information processing device 100 calculates a reference solution u-ₖ* of the insulin dosage using a constraint condition defined by the following formula (15) and a constraint condition defined by the following formula (16), so as to minimize an objective function indicated by the following formula (14), for the representative model. The following formula (16) indicates the constraint condition used to control a blood sugar level y-ₖ of the subject so as to cause the blood sugar level yₖ of the subject to be equal to or more than 80, in consideration of the deviation y˜ₖⁱ of the blood sugar level. Here, a numerical value solution uₖ* of the insulin dosage at the time point k is defined by the following formula (17) using the reference solution u-ₖ*. Here, it is sufficient for the information processing device 100 to perform mathematical optimization once regardless of the number of the forecast scenarios f.

$${min}_{{\overline{u}}_{0} , … , {\overline{u}}_{N - 1}} {{\sum }^{N}}_{k = 1} {\overline{x}}_{k}^{T} \overline{Q} {\overline{x}}_{k} + {\overline{u}}_{k}^{T} R {\overline{u}}_{k} + {\overline{x}}_{N + 1}^{T} {\overline{S}}_{f} {\overline{x}}_{N + 1} \qquad ( 14 )$$

x̅ₖ₊₁=Ax̅ₖ+Būₖ+B_{w}f̅ₖ+D, (15)

yₖ+min{\tilde{y}ₖ¹,\tilde{y}ₖ²,\tilde{y}ₖ³,\tilde{y}ₖ⁴,}≥80 (16)

u₀*=ū₀*+0,u₁ⁱ*=ū₁ⁱ*+ũ₁ⁱ*,u₂ⁱ*=ū₂ⁱ*+ũ₂ⁱ*, (17)

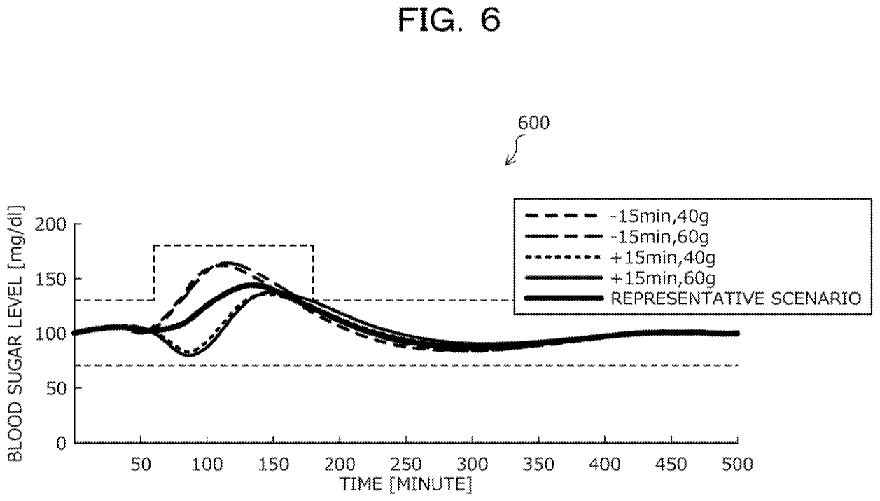

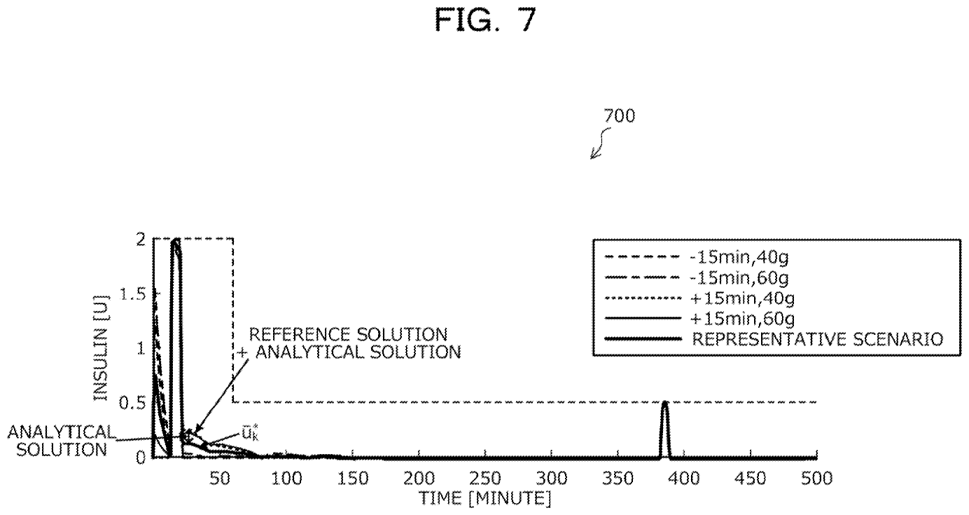

Next, description of FIG. 6 will be made. A graph 600 in FIG. 6 indicates a temporal change of a blood sugar level y-ₖ* corresponding to the reference solution u-ₖ* of the insulin dosage corresponding to the representative scenario. The graph 600 in FIG. 6 also indicates a temporal change of a blood sugar level yₖ* corresponding to the numerical value solution uₖ* of the insulin dosage corresponding to the forecast scenario fⁱ. Next, description of FIG. 7 will be made.

A graph 700 in FIG. 7 indicates a temporal change of the reference solution u-ₖ* of the insulin dosage corresponding to the representative scenario. The graph 700 in FIG. 7 also indicates a temporal change of the numerical value solution uₖ* of the insulin dosage corresponding to the forecast scenario fⁱ.

In this way, as illustrated in the graph 600, the information processing device 100 may cause the blood sugar level yₖ* to be equal to or more than the lower limit value 80 even if any forecast scenario fⁱ is realized. Therefore, the information processing device 100 may avoid life-threatening danger of the subject.

Furthermore, as illustrated in the graph 700, the information processing device 100 may use an appropriate numerical value solution uₖ* of the insulin dosage even if any forecast scenario fⁱ is realized. Since it is sufficient for the information processing device 100 to perform mathematical optimization once, it is possible to reduce a processing load applied when the appropriate numerical value solution uₖ* of the insulin dosage is calculated for each forecast scenario fⁱ.

For example, conventionally, using a constraint condition indicated by the following formula (19) and a constraint condition indicated by the following formula (20) so as to minimize an objective function indicated by the following formula (18), a numerical value solution uₖ* of the insulin dosage is calculated for each forecast scenario fⁱ. In this case, as the number of forecast scenarios fⁱ is larger, the number of times when mathematical optimization is performed increases. Therefore, a processing load applied when the numerical value solution uₖ* of the insulin dosage is calculated increases. Conventionally, for example, in a case where the number of forecast scenarios fⁱ is four, a case is considered where a processing time is 30 seconds.

$${min}_{\underline{{u}_{0}^{( 1 )} , … , {u}_{N - 1}^{( 1 )} , … , {u}_{0}^{( 2 )} , … , {u}_{N - 1}^{( l )}}} {\sum }_{i = 1}^{I} {\sum }_{k = 0}^{N} \underline{{x}_{k}^{( i ) T} {Qx}_{k}^{( i )} + {u}_{k}^{( i ) T} {Ru}_{k}^{( i )} + {x}_{N + 1}^{( i ) T} {S}_{f} {x}_{N + 1}^{( i )}} \qquad ( 18 )$$

xₖ₊₁⁽¹⁾=Axₖ⁽¹⁾+Buₖ⁽¹⁾+B_{w}fₖ⁽¹⁾+D, . . .

xₖ₊₁^{(l)}=Axₖ^{(l)}+Buₖ^{(l)}+B_{w}fₖ^{(l)}+D (19)

minCxₖ⁽ⁱ⁾≥80 (20)

On the other hand, since it is sufficient for the information processing device 100 to perform mathematical optimization once, it is possible to reduce the processing load applied when the appropriate numerical value solution uₖ* of the insulin dosage is calculated for each forecast scenario fⁱ. The information processing device 100 may suppress the processing time, for example, to five seconds.

(Another Operation Example of Information Processing Device 100)

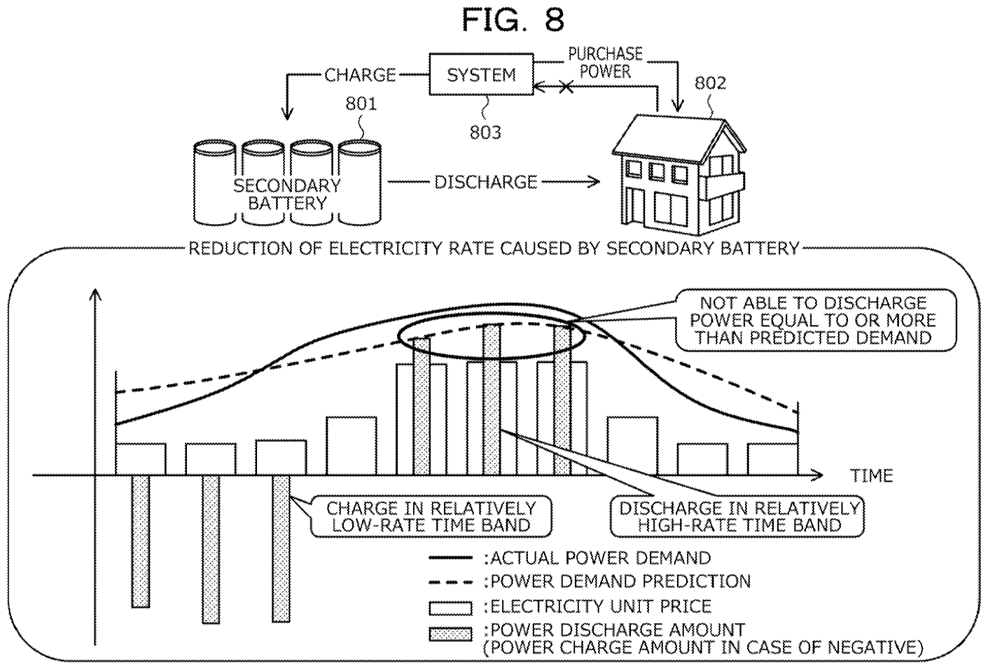

Next, another operation example of the information processing device 100 will be described with reference to FIG. 8. The another operation example corresponds, for example, to a case where the information processing device 100 is applied to the electric field.

FIG. 8 is a diagram illustrating another operation example of the information processing device 100. In FIG. 8, a secondary battery 801 is charged from a system 803 and discharges to a consumer 802. The consumer 802 purchases power from the system 803. Here, it is desirable to control a discharge amount of the secondary battery 801 so as to charge the secondary battery 801 in a time band in which an electricity unit price is relatively low and to discharge from the secondary battery 801 in a time band in which the electricity unit price is relatively high, based on a forecast scenario that predicts power demand.

With respect to this, a maximization problem is set that calculates a discharge amount x of the secondary battery 801 using a constraint condition indicated by the following formula (22), a constraint condition indicated by the following formula (23), and a constraint condition indicated by the following formula (24), so as to maximize an objective function indicated by the following formula (21).

$$max {\sum }_{i = 1}^{H} {P}_{i} {x}_{i} \qquad ( 21 )$$

sᵢ₊₁=sᵢ−xᵢ−fᵢ,i=1, . . . ,H (22)

−U_{c}≤xᵢ≤U_{dc},i=1, . . . ,H (23)

0≤sᵢ≤Uₛ,i=1, . . . ,H+1 (24)

Here, xᵢ represents a discharge amount at a time point i, Pᵢ represents an electricity unit price at the time point i, fᵢ represents power demand at the time point i indicated by a forecast scenario, sᵢ represents a power storage amount at the time point i, U_{c} represents a maximum power charge amount per unit time, U_{dc} represents a maximum discharge amount per unit time, and U_{S} represents a power storage capacity.

Similarly to the operation example illustrated in FIGS. 5 to 7, the information processing device 100 may calculate a numerical value solution for the discharge amount x of the secondary battery 801 by solving the maximization problem described above. As a result, the information processing device 100 may reduce a processing load applied when the numerical value solution for the discharge amount x of the secondary battery 801 is calculated. The information processing device 100 may appropriately control the discharge amount x of the secondary battery 801.

(Overall Processing Procedure)

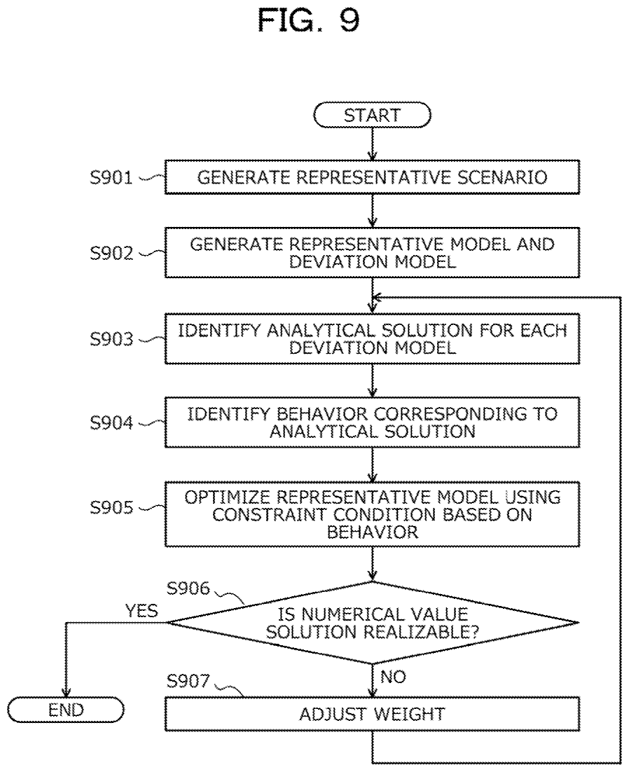

Next, an example of an overall processing procedure performed by the information processing device 100 will be described with reference to FIG. 9. The overall processing is implemented by, for example, the CPU 301, the storage area such as the memory 302 or the recording medium 305, and the network I/F 303 illustrated in FIG. 3.

FIG. 9 is a flowchart illustrating an example of the overall processing procedure. In FIG. 9, the information processing device 100 generates a representative scenario based on a plurality of forecast scenarios (step S901).

Next, the information processing device 100 divides a prediction model and generates a representative model using the representative scenario and a deviation model using a deviation between the representative scenario and the forecast scenario (step S902). Then, the information processing device 100 identifies an analytical solution u-ₖ* for each generated deviation model (step S903). The analytical solution u-ₖ* is, for example, a deviation of an insulin dosage.

Next, the information processing device 100 identifies a behavior y˜ₖⁱ corresponding to the identified analytical solution u-ₖ* (step S904). The behavior y˜kⁱ is, for example, a deviation of a blood sugar level. Then, the information processing device 100 optimizes the generated representative model using a constraint condition based on the identified behavior y˜ₖⁱ and calculates a numerical value solution (step S905). The numerical value solution is, for example, an insulin dosage.

Next, the information processing device 100 determines whether or not the calculated numerical value solution is realizable (step S906). In a case where the numerical value solution is not realizable (step S906: No), the information processing device 100 adjusts a weight applied to a variable to be an input (step S907), and returns to the processing in step S903. The weight is, for example, a coefficient matrix R. In a case where the numerical value solution is realizable (step S906: Yes), the information processing device 100 ends the overall processing.

As described above, according to the information processing device 100, it is possible to generate the representative scenario representing the plurality of forecast scenarios, based on the plurality of forecast scenarios that defines the future value of the first variable. According to the information processing device 100, it is possible to generate the representative model that enables to calculate the future value of the third variable, using the value of the first variable defined by the generated representative scenario and the value of the second variable for control. According to the information processing device 100, it is possible to generate the deviation model indicating the deviation between the prediction model that enables to calculate the future value of the third variable and the representative model, using the value of the first variable defined by the forecast scenario and the value of the second variable, for each forecast scenario. According to the information processing device 100, it is possible to identify the mathematical expression that enables to calculate the analytical solution for the deviation of the second variable, using the deviation model, so as to optimize the value of the first objective variable based on the deviation of the third variable, for each generated deviation model. According to the information processing device 100, it is possible to calculate the reference solution for the value of the second variable, using the representative model and the constraint condition based on the deviation of the third variable corresponding to the analytical solution that may be calculated from each mathematical expression, so as to optimize the value of the second objective variable based on the value of the third variable. According to the information processing device 100, it is possible to calculate the numerical value solution for the value of the second variable, based on the calculated reference solution and the analytical solution that may be calculated from each mathematical expression. As a result, the information processing device 100 may reduce the processing load applied when the numerical value solution for the value of the second variable is calculated while satisfying the constraint condition.

According to the information processing device 100, it is possible to generate the representative scenario that defines the average value of the first variables in the plurality of forecast scenarios, based on the value of the first variable defined by each forecast scenario. As a result, if the value of the first variable is the change amount of the blood sugar level of the subject and the value of the second variable is the insulin dosage, the information processing device 100 may enable to calculate the reference solution for the insulin dosage so that the blood sugar level of the subject does not rise too much.

According to the information processing device 100, it is possible to generate the representative scenario that defines the minimum value of the first variables of the plurality of forecast scenarios, based on the value of the first variable defined by each forecast scenario. As a result, the information processing device 100 may easily satisfy the constraint condition. If the value of the first variable is the change amount of the blood sugar level of the subject and the value of the second variable is the insulin dosage, the information processing device 100 may enable to calculate the reference solution for the insulin dosage so that it is difficult for the blood sugar level of the subject to fall below the lower limit value.

According to the information processing device 100, it is possible to adopt the change amount of the blood sugar level of the subject according to meals as the value of the first variable. According to the information processing device 100, it is possible to adopt the insulin dosage to the subject as the value of the second variable. According to the information processing device 100, it is possible to adopt the difference between the reference value and the blood sugar level of the subject as the value of the third variable. According to the information processing device 100, it is possible to adopt the target to be minimized, including the square of the deviation of the difference, as the value of the first objective variable. According to the information processing device 100, it is possible to adopt the target to be minimized, including the square of the difference, as the value of the second objective variable. According to the information processing device 100, it is possible to adopt that the blood sugar level of the subject that is obtained by adding the reference of the blood sugar level of the subject corresponding to the reference solution and the minimum value of the deviation of the blood sugar level of the subject corresponding to the analytical solution that may be calculated from each mathematical expression is equal to or more than the lower limit value, as the constraint condition. As a result, the information processing device 100 may be applied to the medical field and control the insulin dosage.

According to the information processing device 100, it is possible to adopt the power demand amount as the value of the first variable. According to the information processing device 100, it is possible to adopt the power storage amount as the value of the second variable. According to the information processing device 100, it is possible to adopt the power charge amount as the value of the third variable. According to the information processing device 100, it is possible to adopt the target to be maximized including a deviation of a reduction in an electricity rate as the value of the first objective variable. According to the information processing device 100, it is possible to adopt the target to be maximized, including the reduction in the electricity rate, as the value of the second objective variable. According to the information processing device 100, it is possible to adopt that the value obtained by adding the reference of the power charge amount corresponding to the reference solution and the minimum value of the deviation of the power charge amount corresponding to the analytical solution that may be calculated from each mathematical expression is equal to or more than the lower limit value, as the constraint condition. As a result, the information processing device 100 may be applied to the electric field and may control the power storage amount.

Note that, the information processing method described in the present embodiment may be implemented by executing a program prepared in advance on a computer such as a personal computer (PC) or a workstation. The information processing program described in the present embodiment is executed by being recorded on a computer-readable recording medium and being read from the recording medium by the computer. The recording medium is a hard disk, a flexible disk, a compact disc (CD)-ROM, a magneto-optical disc (MO), a digital versatile disc (DVD), and the like. Furthermore, the information processing program described in the present embodiment may be distributed via a network such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an information processing program for causing a computer to execute a process, the process comprising:
generating a representative scenario that represents a plurality of forecast scenarios that defines a future value of a first variable, based on the plurality of forecast scenarios;
generating a representative model that enables to calculate a future value of a third variable by using a value of the first variable that is defined by the generated representative scenario and a value of a second variable for control;
generating, for each of the plurality of forecast scenarios, a deviation model that represents a deviation between the representative model and a prediction model that enables to calculate the future value of the third variable by using the value of the first variable that is defined by each of the plurality of forecast scenarios and the value of the second variable;
identifying, for each generated deviation model, a mathematical expression that enables to calculate an analytical solution for a deviation of the second variable, by using each generated deviation model, so as to optimize a value of a first objective variable based on a deviation of the third variable;
calculating a reference solution for the value of the second variable, by using the generated representative model and a constraint condition based on the deviation of the third variable that corresponds to an analytical solution that is able to be calculated from each identified mathematical expression, so as to optimize a value of a second objective variable based on the value of the third variable; and
calculating a numerical value solution for the value of the second variable, based on the calculated reference solution and the analytical solution that is able to be calculated from each identified mathematical expression.

2. The non-transitory computer-readable recording medium according to claim 1, the process further comprising:
generating the representative scenario that defines an average value of first variables in the plurality of forecast scenarios, based on the value of the first variable defined by each of the plurality of forecast scenarios.

3. The non-transitory computer-readable recording medium according to claim 1, the process further comprising:
generating the representative scenario that defines a minimum value of first variables in the plurality of forecast scenarios, based on the value of the first variable defined by each of the plurality of forecast scenarios.

4. The non-transitory computer-readable recording medium according to claim 1, wherein
the value of the first variable is a change amount of a blood sugar level of a subject according to meals,
the value of the second variable is an insulin dosage to the subject,
the value of the third variable is a difference between a reference value and the blood sugar level of the subject,
the value of the first objective variable is a target to be minimized and includes a square of a deviation of the difference,
the value of the second objective variable is a target to be minimized and includes a square of the difference, and
the constraint condition indicates that the blood sugar level of the subject is equal to or more than a lower limit value, the blood sugar level of the subject being obtained by adding a reference of the blood sugar level of the subject, which corresponds to the reference solution, and a minimum value of a deviation of the blood sugar level of the subject, which corresponds to an analytical solution that is able to be calculated from each mathematical expression.

5. The non-transitory computer-readable recording medium according to claim 1, wherein
the value of the first variable is a power demand amount,
the value of the second variable is a power storage amount,
the value of the third variable is a power charge amount,
the value of the first objective variable is a target to be maximized and includes a deviation of a reduction in an electricity rate,
the value of the second objective variable is a target to be maximized and includes the reduction in the electricity rate, and
the constraint condition indicates that a total value is equal to or more than a lower limit value, the total value being obtained by adding a reference of the power charge amount, which corresponds to the reference solution, and a minimum value of a deviation of the power charge amount, which corresponds to the analytical solution that is able to be calculated from each mathematical expression.

6. An information processing method, comprising:
generating, by a computer, a representative scenario that represents a plurality of forecast scenarios that defines a future value of a first variable, based on the plurality of forecast scenarios;
generating a representative model that enables to calculate a future value of a third variable by using a value of the first variable that is defined by the generated representative scenario and a value of a second variable for control;
generating, for each of the plurality of forecast scenarios, a deviation model that represents a deviation between the representative model and a prediction model that enables to calculate the future value of the third variable by using the value of the first variable that is defined by each of the plurality of forecast scenarios and the value of the second variable;
identifying, for each generated deviation model, a mathematical expression that enables to calculate an analytical solution for a deviation of the second variable, by using each generated deviation model, so as to optimize a value of a first objective variable based on a deviation of the third variable;
calculating a reference solution for the value of the second variable, by using the generated representative model and a constraint condition based on the deviation of the third variable that corresponds to an analytical solution that is able to be calculated from each identified mathematical expression, so as to optimize a value of a second objective variable based on the value of the third variable; and
calculating a numerical value solution for the value of the second variable, based on the calculated reference solution and the analytical solution that is able to be calculated from each identified mathematical expression.

7. An information processing device, comprising:
a memory; and
a processor coupled to the memory and the processor configured to:
generate a representative scenario that represents a plurality of forecast scenarios that defines a future value of a first variable, based on the plurality of forecast scenarios;
generate a representative model that enables to calculate a future value of a third variable by using a value of the first variable that is defined by the generated representative scenario and a value of a second variable for control;
generate, for each of the plurality of forecast scenarios, a deviation model that represents a deviation between the representative model and a prediction model that enables to calculate the future value of the third variable by using the value of the first variable that is defined by each of the plurality of forecast scenarios and the value of the second variable;
identify, for each generated deviation model, a mathematical expression that enables to calculate an analytical solution for a deviation of the second variable, by using each generated deviation model, so as to optimize a value of a first objective variable based on a deviation of the third variable;
calculate a reference solution for the value of the second variable, by using the generated representative model and a constraint condition based on the deviation of the third variable that corresponds to an analytical solution that is able to be calculated from each identified mathematical expression, so as to optimize a value of a second objective variable based on the value of the third variable; and
calculate a numerical value solution for the value of the second variable, based on the calculated reference solution and the analytical solution that is able to be calculated from each identified mathematical expression.
